(19) United States
(12) Patent Application Publication
(10) Pub. No.: US 2010/0120134 A1
(43) Pub. Date: May 13, 2010

(54) MICRO-ORGANISM PRODUCTION APPARATUS AND SYSTEM

(75) Inventor: Jonathan L. Gal, Rockwall, TX (US)

Correspondence Address: BURNS & LEVINSON, LLP, 125 SUMMER STREET, BOSTON, MA, 02110, US

(73) Assignee: Texas Clean Fuels, Inc., Rockwall, TX (US)

(21) Appl. No.: 12/688,147

(22) Filed: Jan. 15, 2010

Related U.S. Application Data
(63) Continuation-in-part of application No. 12/035,891, filed on Feb. 22, 2008.
(60) Provisional application No. 61/146,910, filed on Jan. 23, 2009.
(60) Provisional application No. 61/021,700, filed on Jan. 17, 2008.
(60) Provisional application No. 60/971,036, filed on Sep. 10, 2007.
(60) Provisional application No. 60/950,731, filed on Jul. 19, 2007.

(51) Int. Cl. C12M 1/00

(52) U.S. Cl. 435/289.1

(57) ABSTRACT

A micro-organism production system having at least one micro-organism growth unit for maintaining therein micro-organisms in solution. Upon the receipt of a predetermined amount of live organisms, liquid and nutrients within the at least one micro-organism growth unit and the application of radiation thereto, the system produces the rapid growth of micro-organisms within the at least one micro-organism growth unit.

### CROSS REFERENCE TO RELATED APPLICATIONS

This application claims priority of provisional application Ser. No. 61/146,910 entitled MICRO-ORGANISM PRODUCTION SYSTEM AND METHOD filed Jan. 23, 2009 and also is a continuation-in-part of copending U.S. patent application Ser. No. 12/035,891 entitled MICRO-ORGANISM PRODUCTION SYSTEM AND METHOD filed Feb. 22, 2008 which claims priority of provisional application Ser. No. 61/021,700 filed Jan. 17, 2008; provisional application Ser. No. 60/971,036 filed Sep. 10, 2007; and provisional application Ser. No. 60/950,731 filed Jul. 19, 2007; all of the above applications being incorporated in their entirety for all purposes.

### BACKGROUND

Over the past 5-10 years, the acceptance and use of ethanol and biodiesel have grown dramatically in the U.S. In 2006, ethanol consumption in U.S. vehicles reached nearly 5 billion gallons, and biodiesel consumption is estimated at about 1 billion gallons. Together, these alternative fuels accounted for about 3% of our nation's total crude oil consumption, most of which comes from other countries. In contrast to the large amounts of foreign crude oil imported into the US, the ethanol and biodiesel used in the U.S. were produced in the U.S. using farm crops (corn and soybeans) as a feedstock. These alternative fuels—known as “biofuels” because they are made from living materials - also have environmental benefits; and it is relatively easy for car manufacturers to produce ethanol and biodiesel vehicles, as the engines and fuel systems for such vehicles are very similar to traditional gasoline and diesel vehicles. Clearly, ethanol and biodiesel are very valid alternatives as fuels for our nation's vehicles.

However, a problem with these new fuels is emerging, as the volumes of their use grow. The problem is simply that there is not enough farmland in the U.S. to supply the quantities of soybeans and corn needed to replace the U.S. demand for crude oil and its Derivatives like gasoline and diesel fuel. Even today, with only 3% of U.S. crude oil demand being supplied by ethanol and biodiesel, the prices of corn and soybeans have skyrocketed, and concerns about food price inflation driven by ethanol demand for corn are mounting.

Micro-Organisms like algae and bacteria offer a potential solution to this problem. For example, photosynthetic micro algae, which are commonly known as “pond scum” and/or “red tide,” are single celled living organisms that consume carbon dioxide, water, sunlight, and nutrients as they grow. A colony of micro algae, after being dried, can be broken down into three types of materials: starch, oil, and protein, using existing technologies. The starch component, like corn starch, can then be further processed into ethanol fuel, using existing technologies. The oil component, like crude oil, can then be processed into biodiesel fuel or other commodities, using existing technologies. In addition, the protein component can be used as livestock feed or fertilizer, using existing technologies. Other commercially important commodities can also be derived from algae and its components, including but not limited to, plastic resins, human nutritional supplements, and food alternatives.

Algae and other micro-organisms can be produced economically in large quantity on much less land than that required by corn and soybeans. Estimates vary, but it is generally accepted that the per acre yield of biomass from algae can theoretically be at least 10 times greater than corn or soybeans and, with the right equipment, some believe that it yields may eventually be more than 100 times greater than corn or soybeans. Yields of that magnitude offer the possibility that the entire U.S. crude oil supply could eventually be replaced by alternative fuels based on algae, or other photosynthetic micro-organisms, that are grown entirely within the borders of the U.S., without significant disruption to the food industry or the real estate industry. However, efforts to achieve these theoretical yields in practice have run into difficulties. To date problems have been encountered achieving such high yields on a commercial scale profitably, consistently, and reliably.

One of the keys to energy independence lies in developing new equipment, processes, and systems that will enable people to grow and harvest high yields of micro-organisms like photosynthetic algae or bacteria consistently, economically, and reliably.

### SUMMARY

One embodiment of the present invention provides, but is not limited to, a micro-organism production apparatus which includes a substantially rigid support member being of a predetermined height having a first end and a second end, the member being made of a material that permits radiation to pass there through, a tubular growth structure circumscribing the support member and capable of maintaining therein micro-organisms in solution, the tubular structure capable of permitting radiation to pass there through, and the tubular structure having a length substantially greater than the predetermined height of the support member; a radiation transmitting component adjacent at least the first end of the support member capable of directing incoming radiation onto the tubular growth structure; and a reflecting structure circumscribing the tubular growth structure and located adjacent the second end of the support member capable of redirecting radiation towards the tubular growth structure; wherein upon the receipt of a predetermined amount of live organisms, liquid and nutrients within the tubular growth structure and the application of radiation through the radiation transmitting component, the apparatus produces the rapid growth of micro-organisms within the growth structure.

The tubular growth structure may be wound around the support member. The micro-organism production apparatus may further include a filtering component being adjacent to or incorporated as part of the radiation transmitting component capable of permitting predetermined wavelengths of radiation to pass onto the tubular growth structure. The support member and the growth structure may be made of transparent material. The radiation transmitting component may be a Fresnel lens, a diffractive or refractive element or a holographic element.

Another embodiment of the present invention provides, but is not limited to, a micro-organism production apparatus includes a substantially tubular, coiled growth structure made of a substantially rigid material capable of maintaining therein micro-organisms in solution, the tubular structure having a first end and a second and capable of permitting radiation to pass there through.

Yet another embodiment of the present invention provides, but is not limited to, a micro-organism production apparatus including a substantially a substantially rigid support member having a first end and a second end, the member being made of a material that permits radiation to pass there through; a reflecting structure circumscribing the support member and located adjacent the second end of the support member to form a growth cavity there between capable of maintaining therein micro-organisms in solution.

Still another embodiment of the present invention provides but is not limited to, a micro-organism production system including a substantially rigid support member being of a predetermined height having a first end and a second end, the member being made of a material that permits radiation to pass there through; micro-organism growth means for maintaining therein micro-organisms in solution; a radiation transmitting component adjacent at least the first end of the support member capable of directing incoming radiation onto the micro-organism growth means; a reflecting structure circumscribing the micro-organism growth means and located adjacent the second end of the support member capable of redirecting radiation towards the micro-organism growth means; means for providing carbon dioxide interconnected to a first end of the micro-organism growth means; a two way valve and a pump interposed between the means for providing carbon dioxide and the first end of the micro-organism growth means; means for providing live organisms and a source of liquid and nutrients interconnected to a second end of the micro-organism growth means; and another two way valve and another pump interposed between the means for providing live organisms and the source of liquid and nutrients and the second end of the micro-organism growth means; wherein upon the receipt of a predetermined amount of the live organisms, liquid and nutrients within the micro-organism growth means and the application of radiation through the radiation transmitting component, the system produces the rapid growth of micro-organisms within the growth means.

The micro-organism growth means and the support member may be a substantially rigid, coiled tubular member, it may be a coiled tubular member circumscribing the support member, or it may be a growth cavity between the support member and the reflecting structure. The micro-organism growth means may include a plurality of micro-organism growth units. The micro-organism production system may further include a housing substantially encompassing the plurality of micro-organism growth units. The micro-organism production system may further include a system for positioning the plurality of growth units in a pre-selected direction. The micro-organism production system may further include means for controlling the feeding of live micro-organisms in the micro-organism growth means. The micro-organism production system may further include means for providing an auxiliary source of energy from heat generated within the housing.

A further embodiment of the micro-organism growth apparatus , but not limited thereto includes an inner member having a first end and a second end, wherein the inner member being made of a material that permits radiation to pass there through, an outer structure circumscribing the inner member, the outer structure having a predetermined configuration facing said inner member, and the predetermined configuration being in the form of protrusions and indentations, a growth cavity formed between the inner member and the outer structure capable of maintaining within said growth cavity micro-organisms in solution, a radiation transmitting component adjacent at least the first end of said inner member capable of directing incoming radiation onto said growth cavity, and a reflective component adjacent at least said second end of said inner member. In addition, the inner member may have protrusions and indentations on a surface facing the growth cavity.

Further, but not limited thereto, the protrusions and indentations substantially increase the surface area of the inner member and the outer structure. Additionally, but not limited thereto, the inner member and outer member may be made of material which allows the passage of radiation therethrough. In a further embodiment a plurality of micro-organism growth apparatus or growth units are located within a housing and the housing may have reflective sides and bottom to direct radiation to pass into the growth cavity.

An even further embodiment of the present invention provides, but is not limited to, a method of producing micro-organisms which includes the steps of, but not limited to providing live organisms, water and nutrients into at least one transparent micro-organism growth unit; providing a source of radiation and directing the source of radiation to the at least one growth unit, the at least one growth unit may be substantially rigid and substantially vertically upright and being of substantial length; filtering the radiation such that only a preselected wavelength of the radiation reaches the at least one micro-organism growth unit; providing CO₂ to the growth unit; permitting growth of micro-organisms within the at least one growth unit until the growth unit becomes substantially opaque or until a predetermined time interval has occurred; stopping the provision of CO₂ to the at least one growth unit; harvesting the micro-organisms from the at least one growth unit; removing any excess liquid from the harvested micro-organisms; and recycling the excess liquid back into the system for future use.

These and further embodiments are described in greater detail herein below; and for a better understanding of the present invention, reference is made to the accompanying drawings and detailed description and its scope will be pointed out in the appended claims.

### BRIEF DESCRIPTION OF THE DRAWINGS

FIG. 1 is a schematic overview of the Micro-Organism production system (MOPS) of this invention;

FIG. 2 is a cross-sectional view of a single MOPS “growth unit” of this invention;

FIG. 3a is a pictorial, exploded view of a single MOPS “growth unit” of this invention;

FIG. 3b is a cross-section of a single MOPS “growth unit” of this invention;

FIG. 4 is a pictorial, exploded view of eight (8) growth units assembled into a framing & housing system of this invention;

FIGS. 5-8 illustrate schematic diagrams of four main phases of operation of the MOPS of this invention;

FIG. 9 is a cross-sectional view of a further embodiment of a single MOPS growth unit of this invention;

FIG. 10a is a pictorial, exploded view of the growth unit of this invention depicted in FIG. 9;

FIG. 10b is a pictorial, cross-sectional view of the growth unit of this invention represented in FIGS. 9 & 10a;

FIG. 11 is a cross-sectional view of another embodiment of a single growth unit of this invention;

FIG. 12a is a pictorial, exploded view of the growth unit of this invention, which is depicted in FIG. 11l;

FIG. 12b is a pictorial, cross-sectional view of the growth unit of this invention, which is represented in FIGS. 11 & 12a;

FIG. 13 is a top view of still another embodiment of a single growth unit of this invention;

FIG. 14a is a pictorial, exploded view of the growth unit of this invention, which is depicted in FIG. 13;

FIG. 14b is a pictorial, cross-sectional view of the growth unit of this invention, which is represented in FIGS. 13 & 14a;

FIG. 15 is a pictorial, exploded view of the housing & framing of the MOPS of this invention depicted in FIG. 9;

FIG. 16 is a pictorial view of the housing & framing of the MOPS of this invention depicted in FIG. 11;

FIG. 17a is a plan view of a MOPS “installation” of this invention;

FIG. 17b is a pictorial, exploded view of two manifold connections that connect MOPS growth units together in a MOPS array of this invention;

FIG. 18a is a plan view of the MOPS of this invention as depicted in FIG. 9 installation;

FIG. 18b is pictorial, exploded view of two manifold connections that connect MOPS growth unit of FIG. 18a together in a MOPS array of this invention;

FIG. 19 is another embodiment of a MOPS installation of this invention;

FIG. 20 is still another embodiment of the framing and housing of a MOPS installation;

FIG. 21 is still a further embodiment of a single MOPS growth unit of this invention;

FIG. 22a is a pictorial, exploded view of a single MOPS growth unit of this invention, which is depicted in FIG. 21;

FIG. 22b is a pictorial, exploded, cross-sectional drawing of the single MOPS growth unit of this invention, which is depicted in FIGS. 21 & 22a;

FIG. 23a is a pictorial, exploded view of another embodiment of a single MOPS growth unit;

FIG. 23b is a pictorial, exploded, cross-sectional view of the single MOPS growth unit depicted in FIG. 23a;

FIG. 24a is a pictorial, exploded view of the single MOPS growth unit;

FIG. 24b is a pictorial, exploded, cross-sectional view of the single MOPS growth unit of this invention, as depicted in FIG. 24a;

FIG. 25 is a top view of another alternative embodiment of the MOPS of this invention;

FIG. 26 is a top view of yet another alternative embodiment of the MOPS of this invention;

FIG. 27 is a top view of yet another alternative embodiment of the MOPS of this invention;

FIGS. 28a & 28b represent an embodiment of a wall panel of the MOPS housing of this invention;

FIGS. 29a & 29b represent an alternative embodiment of a wall panel of the MOPS housing of this invention;

FIGS. 30a, 30b, & 30c represent yet another alternative embodiment of a wall panel of the MOPS housing of this invention; and

FIGS. 31a and 31b, 32 and 33 are pictorial representations of still another embodiment of a single MOPS growth unit of this invention.

### DETAILED DESCRIPTION OF THE INVENTION

The present invention may be understood by the following detailed description, which should be read in conjunction with the attached drawings. The following detailed description of certain embodiments is by way of example only and is not meant to limit the scope of the present invention.

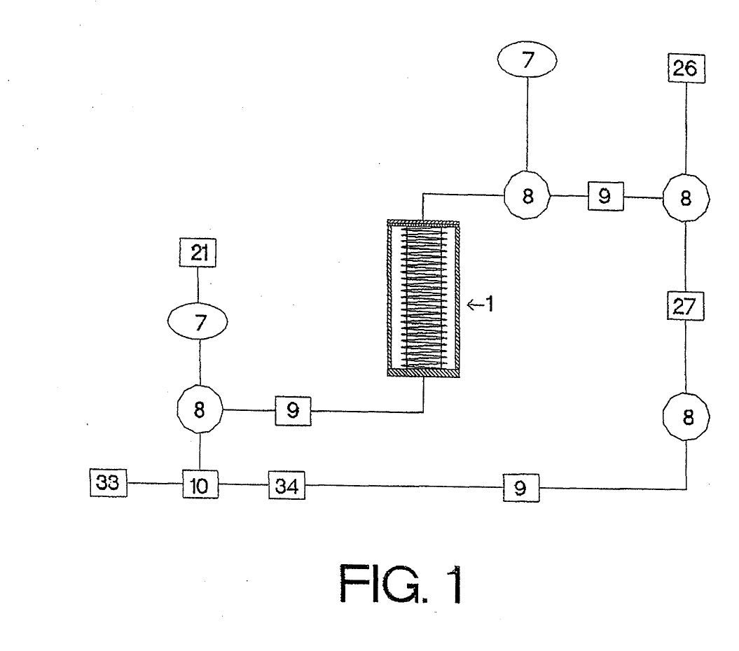

A schematic overview of the Microorganism Production System (MOPS) invention is shown in FIG. 1. The MOPS is a system designed to harness the natural process of photosynthesis, which is used by plants and other photosynthetic organisms, to produce algae and other microorganisms on a commercial scale which can then be converted, using other equipment and processes, into ethanol & biodiesel, two alternative fuels that are gaining market acceptance rapidly in the US as well as other parts of the world.

In addition, the microorganisms produced by this system may have other uses in the energy, fuel, and food industries. For example, dried micro organisms may be useful as a feedstock for various “gasification” technologies that are being used and developed for the production of electricity. They may also be burned directly (rather than being converted to ethanol & biodiesel before burning) in electricity generation or heat production.

Furthermore, the micro organisms produced by this invention may be convertible into “cellulosic ethanol” using entirely new processes that are currently being developed in that arena. In addition, the system can also be used to produce food quality microorganisms for use in the human & animal food, vitamin, & supplement industries. In addition, finally, the microorganism produced by this system may have applications, which are not contemplated here or developed yet by anyone at this time. This patent application covers all possible applications of the MOPS.

The MOPS is designed to produce the maximum possible return on investment in whatever application is contemplated. This goal corresponds closely with and incorporates the goal of maximizing yield per acre per year, but it is not precisely the same measure.

Using return on investment as a goal, rather than simply yield per acre, means that the cost of building and operating the equipment is factored into the equations.

Microorganisms like algae & bacteria come in many different varieties, shapes, sizes, and colors. In fact, there are tens of thousands of different known species in existence; and there are probably many more species that have not yet been discovered. In addition, some scientists are developing genetically engineered species of photosynthetic bacteria and algae, which may work well in the MOPS. Each species, whether natural or engineered, has its own unique characteristics and biochemical needs; but in general, photosynthetic micro organisms like algae and bacteria need the following resources in order to grow and reproduce: sunlight, carbon dioxide, water, and nutrients.

In nature, microorganisms such as photosynthetic algae tend to find everything they need in situations like, for example, the surface of a fresh water pond. There, they have access to water & nutrients in the pond, sunlight from the sun, and carbon dioxide in the air. It turns out, however, that the resources available in a fresh water pond are generally far greater than the algae can actually utilize, due to certain natural phenomenon that restrict growth. The MOPS is designed to create a carefully controlled, manmade environment that overcomes these natural inhibitors to the growth of algae and other microorganisms. It regulates temperature, keeping it in the optimum range throughout the year. It filters & diffuses sunlight in a manner that allows for as many algae cells to be irradiated by the preferred wavelengths of radiation as possible, thus maximizing the utilization of sunlight. Water and nutrient flow are precisely controlled and water is recycled, in order to maximize utilization of those resources. Carbon dioxide is injected in a carefully controlled and filtered manner that should also improve the returns on investment of the system. The system is completely sealed and closed other than certain filtered openings, which prevents contamination by unwanted organisms that may inhibit growth of the desired organism. In addition, it is scaleable in a manner that supports cost effective and efficient manufacturing, assembly, operation, and maintenance of the system.

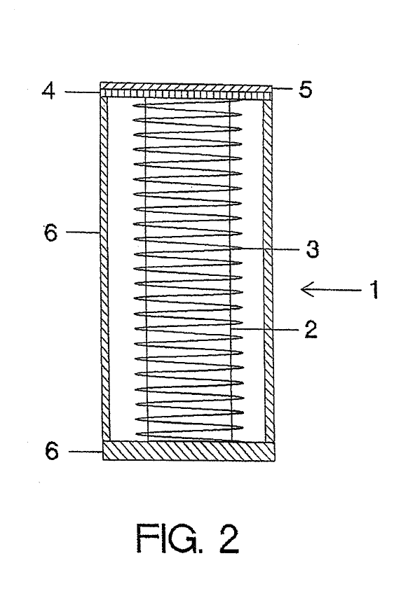

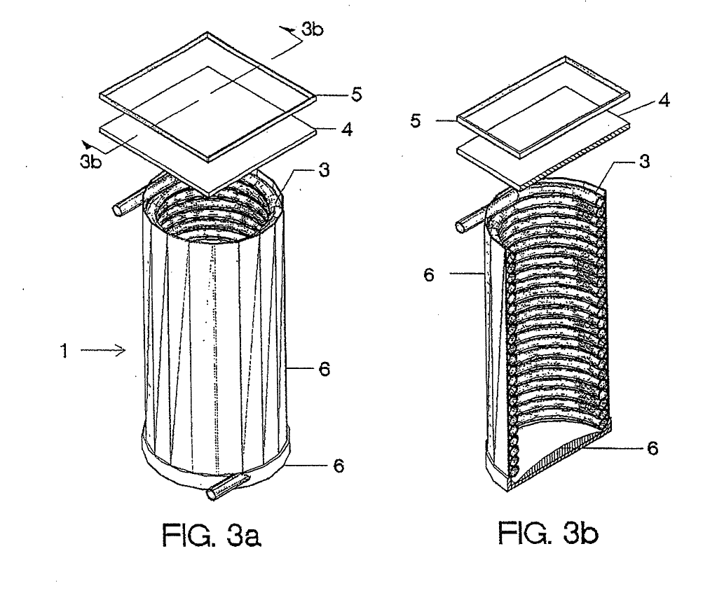

The MOPS is capable of being scaled up to produce commercially useful quantities of fuel. The scaled up MOPS is capable of comprising of hundreds, thousands, tens of thousands, or even more MOPS “growth units” 1. An embodiment of a single MOPS “growth unit” 1 is depicted in FIGS. 2, 3a, and 3b. It should further be realized that the concepts set forth with respect to growth unit 1 apply to other embodiments as well. Further, any source of radiation, natural or artificial, which may be of different wavelengths, may be used with this invention. Therefore, the terms radiation and light may, at times, be interchangeable. More specifically the major components of growth unit 1 are set forth as follows:

The “Support Tube” 2 is a transparent or translucent tube through which radiation can pass easily with minimal loss of radiation. This could be glass, plastic, or any other translucent or transparent material of sufficient strength to hold the other components in the proper positions around it. The tube is erected substantially vertically, although other angles may be used. The vertical may also be used under certain conditions, with one end open to a source of radiation. The height of the tube should be up to 500 times greater than the internal diameter of the tube. The thickness of the tube can be up to several feet in thickness. For cost effectiveness, the thickness should be as small as possible, while still maintaining sufficient strength. For example, but not limited thereto, the support tube 2 can be made of clear polycarbonate approximately 4 feet tall, 18 inches interior diameter, and 1/16^{th} inch thick.

The “Growth Tube” 3 is a translucent or transparent tube, which carries the microorganisms in solution with their necessary nutrients and their supply of gases, including carbon dioxide. In order to ensure that the microorganisms do not shade each other out, this tube 3 should have a fairly narrow diameter, approximately up to 1200 inches. In order to facilitate the maximum transmission of radiation to the growing organisms, this tube should be made of a thin, clear, translucent, or transparent material; and will function most effectively when airtight. In order to maximize the number of organisms within the tube that are exposed to, for example, sunlight, the growth tube 3 should be as long as possible, anywhere up to 1,000,000 times the height of the support tube 2.

The growth tube 3 is wrapped around the support tube 2 in a manner that maximizes the length of the growth tube 3 that contacts or is adjacent to the surface of the support tube 2, so that the utilization of incoming light and radiation is maximized As an example of the growth tube 3, it can have a 2 inch interior diameter, be about 150 feet long and be 1/8th inch thick, although these dimensions are only for purposes of example and not limitation. These dimensions can have the effect of increasing the light utilization significantly, compared to the natural algae pond. Other combinations of dimensions and ratios can also be used. In addition, the growth tube 3 can be made of rigid or substantially rigid material that holds its own shape, in which case the support tube 2 is not necessary. Alternatively, the growth tube 3 can be supported by some sort of framing or other support components, not shown, which would also obviate the need for the support tube 3.

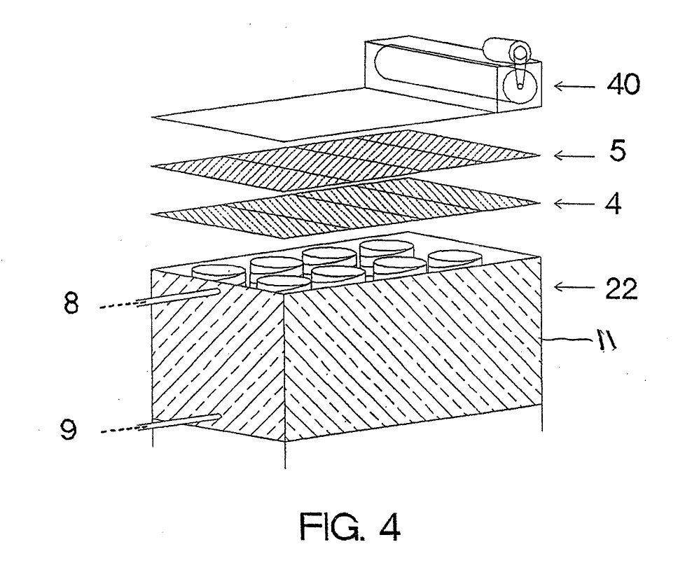

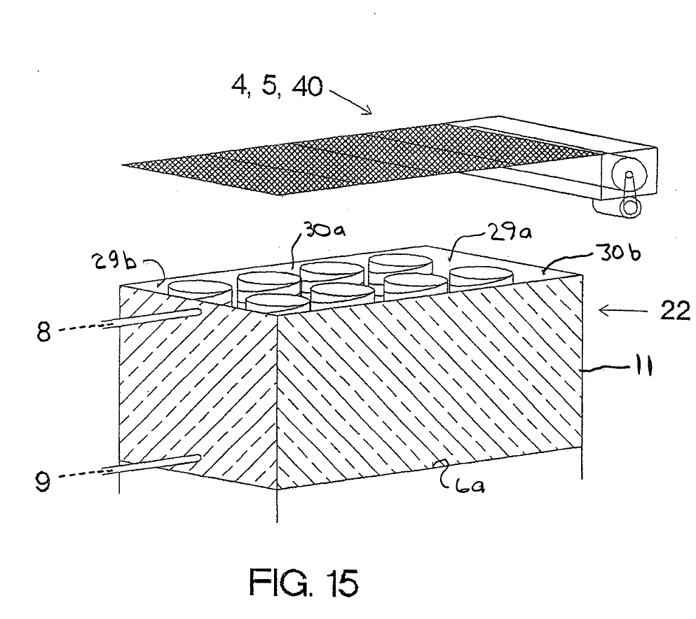

One, or more, “Light/Radiation Diffusing Component” 4 is attached to one open end of the support tube 2, closing the end. The radiation diffusing component 4 can be any one of a number of different kinds of lenses made of glass, plastic, or other materials, which have the effect of spreading, or diffusing, light from a single source like the sun. An example of such a component 4 could be a negative Fresnel lens because it is a low cost lens that diffuses radiation effectively, although the present invention is not limited to just such a lens and can use different or reflective lenses as well. Other lenses, including but not limited to double concave lenses, single concave lenses, double convex lenses, single convex lenses, and custom built lenses built of glass, plastic, or other materials could also be used to spread the incoming sunlight. In addition to various types of lenses, other types of diffusers can be used. For example, some types of paper diffusers or conventional fluorescent light diffusers, which may not be generally thought of per se, as a “lens” can also be used. The radiation diffusing component 4 can also be supported by a framing that is part of the roof of the housing 11, as depicted in FIGS. 4, 15, & 16.

Zero, one, or more, “Radiation Filter(s)” 5 are also attached to the open end of the support tube 2, in order to select the wavelengths of radiation that are most beneficial for the growth of the particular micro-organism to be grown. Since the MOPS can be used for any one of a number of different micro-organisms, which may have different wavelength preferences, any one, or more, of a number of different radiation filters 5, such as, but not limited to a UV filter, can be attached at this point. The radiation filter(s) can also be supported by a framing that is part of the roof of the housing 11, as depicted in FIGS. 4, 15, & 16; and they can also be located on either side of the diffusing component 4 or they can be added as a film or a coating that is adhered to, sprayed, or painted, onto the surface of the radiation diffusing component 4.

One, or more, “Reflecting Surface(s)” 6 are attached at one end or proximate an end of the support tube 2 opposite diffusing component 4 and also around the exterior of the growth tube 3. The purpose of these reflecting surfaces is to capture any “leftover light” that is not utilized by the growing microorganisms on the first pass and reflect it back towards the growth tube 3 so that it can be utilized. The “Reflecting Surface” can be a mirror of any shape or any other reflective surface like a reflective foil. In an embodiment of the invention, a mirror or other Reflective Surface 6 will be fixed to one end of the support tube 2, opposite to the diffusing component 4. In addition, a reflective surface 6 can also be affixed to the interior surface of the housing 11, both of which are other surfaces that may be exposed to “leftover light” or other radiation; and it is desirable to reflect that light and/or radiation back towards the growing microorganisms so that it can be utilized rather than wasted.

Referring to FIG. 1, two “Gas/Air Filters” 7 are attached to either end of the growth tube 3, so that any gas pumped into the growth tube 3 as a source of carbon dioxide, will be as clean as possible, in order to avoid contamination of the system with unwanted organisms. These filters could be any one of numerous different filters currently on the market, but they should be made of a pore size that excludes most, if not all, living organisms, without exclude molecular sized gases. Filters 7 may be of the type typically used for medical & research situations where contamination with unwanted organisms must be prevented, but they should be in an appropriate size for the MOPS.

Still referring to FIG. 1, one, or more, “Two Way Valves & Connectors” 8 are attached at either end of the growth tube 3, in order to control the inputs and outputs coming into and out of the growth tube 3 during different phases of its operation. These should preferably be air tight in all respects, and will preferably coincide with the dimensions of the growth tube 3. It is also possible to use automated valve systems that are driven either by timers or by sensors that detect the appropriate time to switch them.

Still referring to FIG. 1, one, or more, “Pumps, Compressors, & Regulators” 9 are attached to the system at either end of the growth tube 3. Pumps 9 substantially coincide with the inlet dimensions of the growth tube 3. The pump 9 at one end of the system can be a one-way or a two-way pump. That pump 9 will either pump “seed organisms” into the system, or it will pump water & nutrients into the system, depending on how that the valve is set and on which phase of operation the system is functioning. The pumps 9 at the other end of the system are preferably two-way pumps 9, though one of them could be a one way pump 9. Either one will pump CO₂ into the system, or it will suck the finished organism laden solution out of the system for harvest. Making it a two way pump 9 saves having to disconnect and reconnect when switching directions. The other pump 9 at this end, which is preferably a one-way pump 9 will pump “residual water” from the centrifuge back into the water and nutrient chamber for reuse, which allows the system to recycle water thus making it a more profitable and resource efficient system. A wide variety of pumps 9 may suit these purposes, but they should preferably be strong enough to get the job done efficiently and cost effectively. The two way pump 9 that pumps the finished organism laden solution out of the growth tube 3 and into the centrifuge or collection device, in particular, should preferably be strong enough to suck a fairly thick & viscous solution of microorganisms, because the finished, organism laden solution may be—but not necessarily—very thick and viscous, depending on the type of organism grown and on the duration of growth between harvest, the description of which is provided below.

Still referring to FIG. 1, one, or more, “Centrifuges” 10 is attached to the “harvest” line of the system. When the organism laden solution is harvested, it will be made up of microorganisms, leftover water, leftover nutrients, and some leftover gases. This possibly viscous, but very wet and liquid, solution will flow straight into a centrifuge 10, or into some other intermediary collection device(s), before going to the centrifuge, where it will be spun and dried down. The centrifuge 10 spins its contents very fast, causing the water to separate from the other contents. Any one of a large number of conventional centrifuges 10 could be used for this purpose, as long as they are powered appropriately for the size of the MOPS system being operated and for the organism that is being grown in the MOPS system at that time. In some cases, downstream processing technologies like “sonification” may prefer to accept “wet algae” (algae in solution with water prior to drying) as their input rather than dried algae, in which case this drying step and centrifuge 10 can be omitted. After centrifugation, dried algae is collected in a “Harvest Container” 33, while the leftover water solution is channeled into a separate “Collecting Component” 34, from which is can be recycled back into the system for re-use in the next cycle.

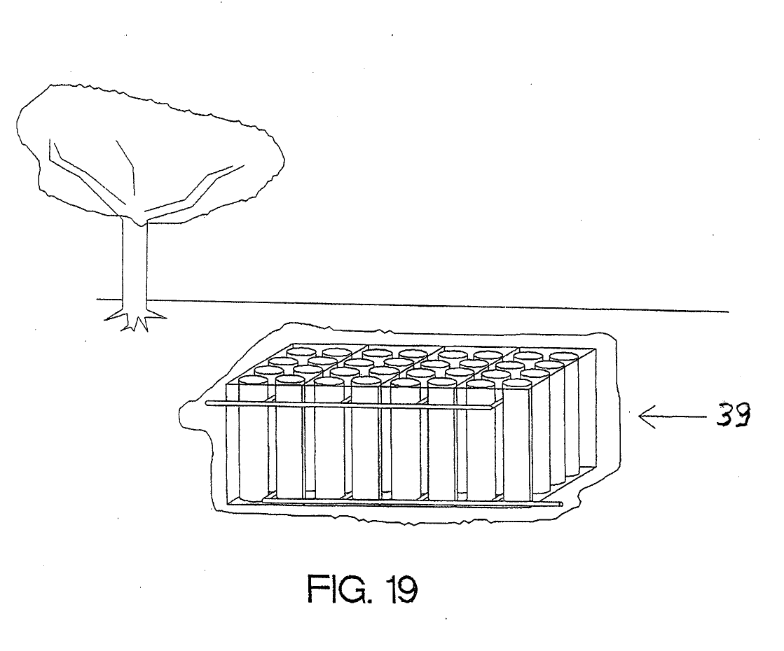

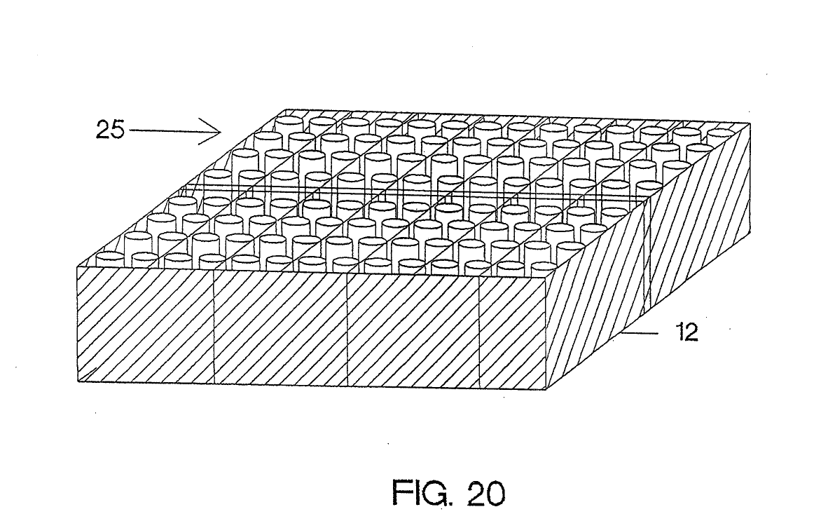

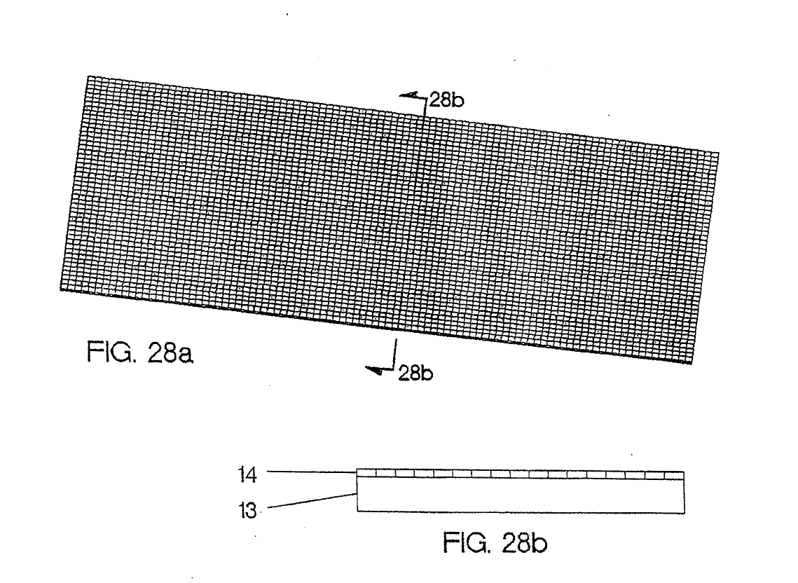

Referring to FIGS. 20, 28a, 28b, 29a, 29b, 30a, 30b and 30c, one, or more, “Housing(s)” 11 where shown, is provided around the units 1 and other parts, in order to (a)maintain a consistent temperature range at all times, (b)prevent wind damage to the units 1 and other parts; (c)provide a support framework for the diffusing component 4, the radiation filter 5, the growth units 1, and other components; and (c)in certain embodiments, to provide a framework on which to affix a reflective surface 6. The side panels 12 of the housing 11 should be of a structural & wind resistant layer 13. In addition, depending on the particular embodiment to be built, the side panels 12 can also consist of an insulative layer 14, a reflective surface 6, and strips of photovoltaic surfaces 15. The housing 11 can also include, depending on the location of the installation 25, a manual or thermostatically controlled heating & cooling system that should be selected for cost effectiveness, not shown.

As shown in FIG. 4, the roof of the housing 11 may be made of 3 layers: the diffusing component 4, the radiation filter 5, and a retractable “Insulative Roof” 40 that can be closed at night to prevent heat loss and opened during the day to allow sunlight to enter. Alternatively, these three layers can be fused into a single layered roof that accomplishes all of the functions with a single layer, as shown in FIG. 15. In yet another embodiment, not shown, a permanent roof that is both insulative and clear can be substituted for the retractable layer of insulation.

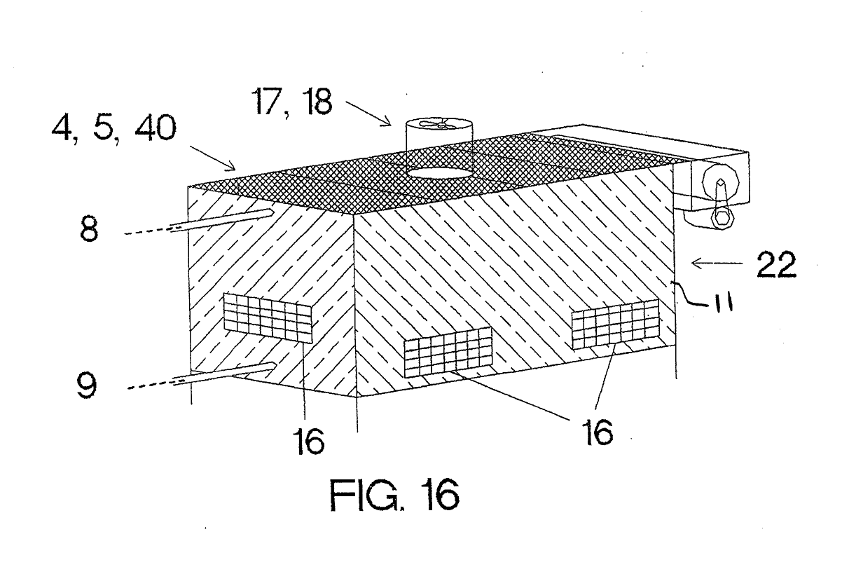

In one embodiment, as shown in FIG. 16, the cooling system of the housing 11 is comprised of one or more low level side air vent(s) 16 on the sides of the housing and one or more chimney vent(s) 17 on the top of the housing. In addition, a wind turbine 18 can be affixed within the chimney vent 17, in order to generate some electricity from the cooling of the housing 11. As the housing 11 cools, a pressure gradient (also known as “wind”) is generated within the housing 11, causing the warm air inside the housing 11 to escape out the chimney vent 17 as cool air enters through the side vents. As the warm air escapes, it turns the wind turbine 18, which generates some electricity. Although electricity generation is not the primary purpose of this invention, the relatively small amounts of electricity generated by a cooling system of this nature may be sufficient to power the pumps 9, valves 8, centrifuge 10, and other electrical components that may be incorporated into the system. Alternatively, electricity generated in this manner can be stored in a battery in order to power the heating system for the housing 11 during colder months of operation.

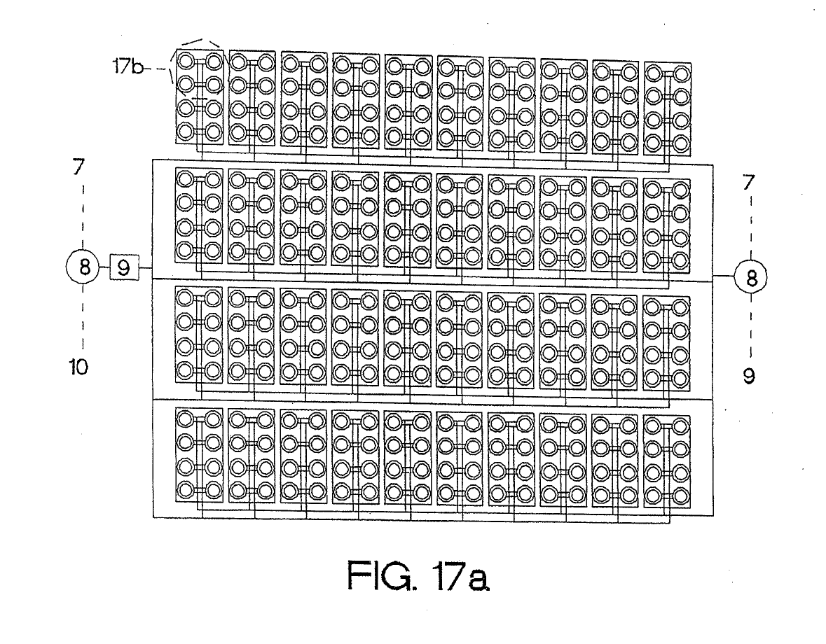

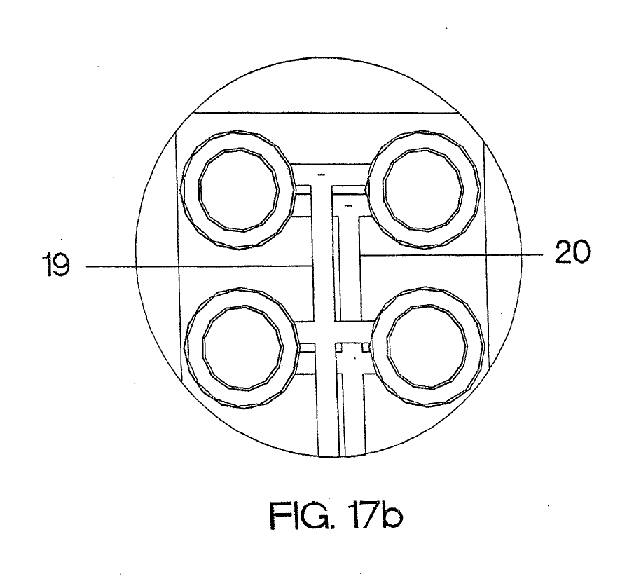

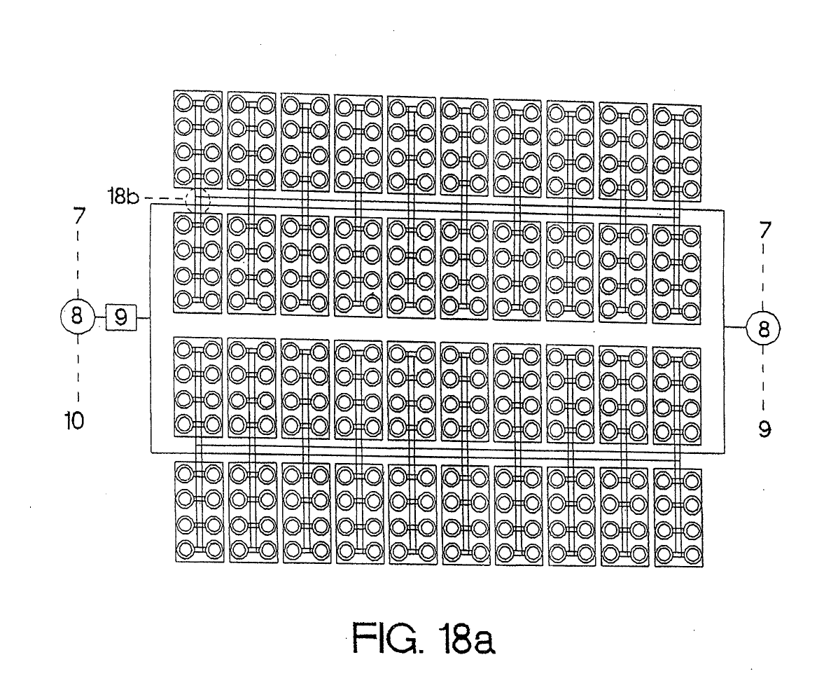

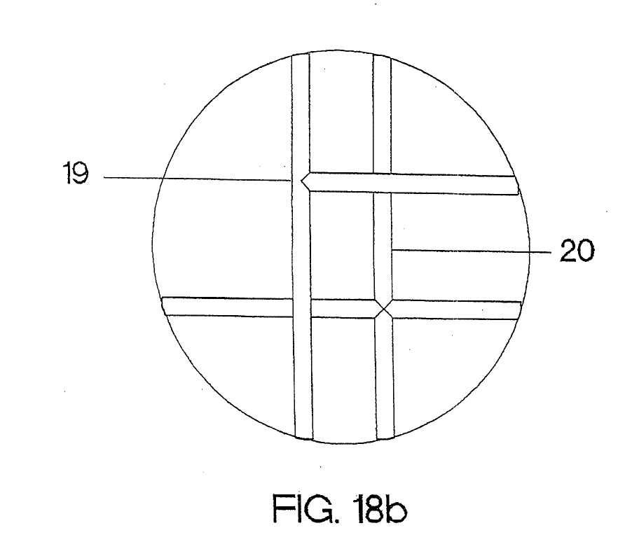

Nutrients, water, and seed stock are fed into a growth unit 1, according to FIGS. 1 & 5-8. As shown in FIGS. 17b and 18b, feeding pipe 19 is also connected to an air vent 21, via a valve 8, which serves to vent the system during certain phases of operation, as shown in FIGS. 1, & 5-8. When a plurality of growth units 1 are connected together, as in a module 22 (see FIG. 15, for example), or when a plurality of modules 22 are connected together, as in a complete installation 25 as shown in FIGS. 16 and 20, the feeding pipes 19 are configured in a manifold configuration, preferably as shown in FIGS. 17a and 17b but can also be configured as shown in FIGS. 18a and 18b, or in other configurations not shown.

Algae are harvested through a drainage pipe 20 as shown in FIGS. 17b and 18b, according to the schematic diagrams in FIGS. 1, and 5-8. This drainage pipe 20 is also connected, via a valve 8, a filter 7, and a pressure regulator 9 to CO₂ source 21. When a plurality of growth units 1 are connected together, as in a module 22, or when a plurality of modules 22 are connected together, as in a complete installation 25, the drainage pipes 20 are configured in a manifold configuration, preferably as shown in FIGS. 17a and 17b, but can also be configured as shown in FIGS. 18a and 18b, or in other configurations not shown.

With growth module 22 as shown in FIG. 4 made up in one embodiment, of eight growth units 1 per module 22, modules 22 can be mass produced in order to supply a wide variety of customer size demands. As pointed out before, these numbers of units are for example and not limitation. A manufacturing and assembly line can be created, in which modules 22 move through the production line just as cars move through mass production lines. Smaller customers can order small numbers of modules 22, while larger customers can order larger numbers of modules 22. By using modular scaling of this nature, all sized customers can benefit from the economies of mass production, which will enable people to utilize this technology on both small and large point sources of CO₂. In addition, when scaling a MOPS installation 25, certain pumps 9, valves 8, and connectors 9 can be combined and connected in parallel or in series and by manifolding. Some suggested methods of connection, though not limiting, are shown in FIGS. 17a, 17b, 18a, and 18b.

Alternatively, the MOPS can be scaled in several other ways. First, it can be scaled such that a plurality of growth units 1 are enclosed in a larger perimeter of housing 11, as shown in FIG. 20. Alternatively, in some locations, a plurality of growth units or modules 22 can be installed underground, as shown in FIG. 19. An underground installation 39 could not only provide insulation and wind protection for the systems, but also some protection against more violent events including but not limited to acts of war, tornadoes, hurricanes, tropical storms, severe thunderstorms, or lightning strikes. If installed below the ground, a MOPS should still be installed with its uppermost components very close to the ground level, so that it does not suffer from shading that could result from installing it far below the surface of the ground.

Various different kinds of conventional support footings, not shown, can be used to situate MOPS modules 22, depending on the preferences of the site owner(s), manager(s), and operator(s). Some options include but are not limited to concrete block footings, concrete slab footings, and also a rail system on which individual MOPS units can be shuttled around the installation 25 quickly and easily for installation, maintenance, or other purposes.

MOPS growth units 1, modules 22, or complete installations 25 can also be mounted on a motorized base that tracks the sun throughout the day, so that it is perfectly aligned with the sun as the sun crosses the sky during the course of the day, which may help to optimize the utilization of sunlight by the system.

Conventional monitoring instrumentation, not shown, could be included in a MOPS installation, including but not limited to: CO₂ sensors, NOₓ sensors, SOₓ sensors, O₂ sensors, thermometers, turbidity sensors, pH meters, and nutrient concentration monitors. Such monitors can also be used as triggers for valves and other components that need to be switched at appropriate times, depending on operating parameters that can be measured with a sensor. In addition, as shown in FIGS. 4, 15, & 16, MOPS modules 22 can be affixed with a motorized, automatic, Insulative Roof 40 that is closed at night to keep the interior of the module 22 warm and opened during the day to allow radiation into the module.

It may also be advantageous to install large mirrors or other reflective surfaces 6 around the periphery of a large MOPS setup, in order to reflect additional solar radiation towards the growth tubes. Other modifications that improve the utility and profitability of larger scale MOPS setups over the single unit setup that is described here are also considered as part of the present invention.

As protection against animals, vandals, terrorists, enemy combatants, or other threats to its integrity, a MOPS installation 25 could also be surrounded by a perimeter security fence, not shown, of appropriate dimensions for the location.

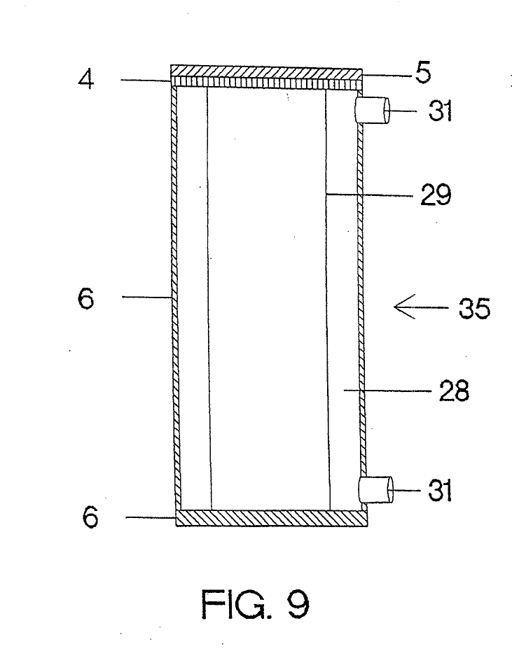

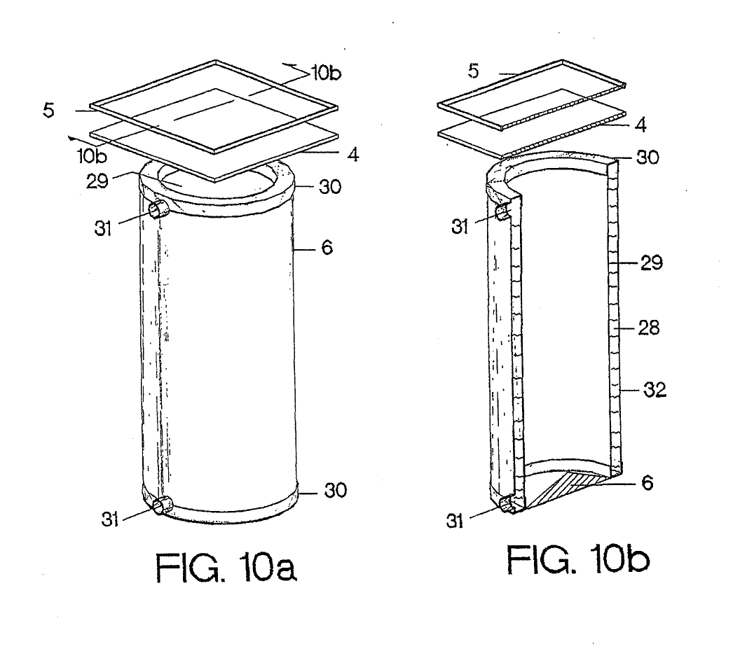

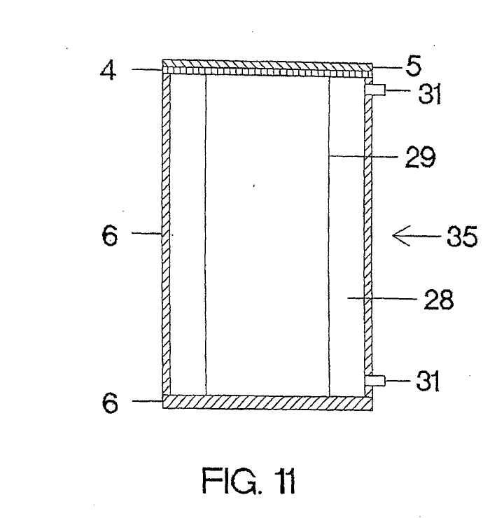

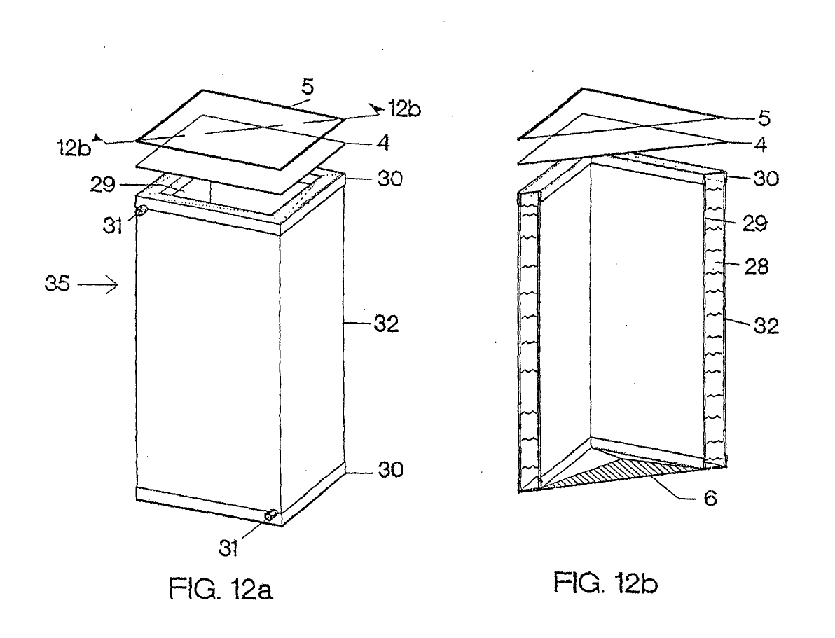

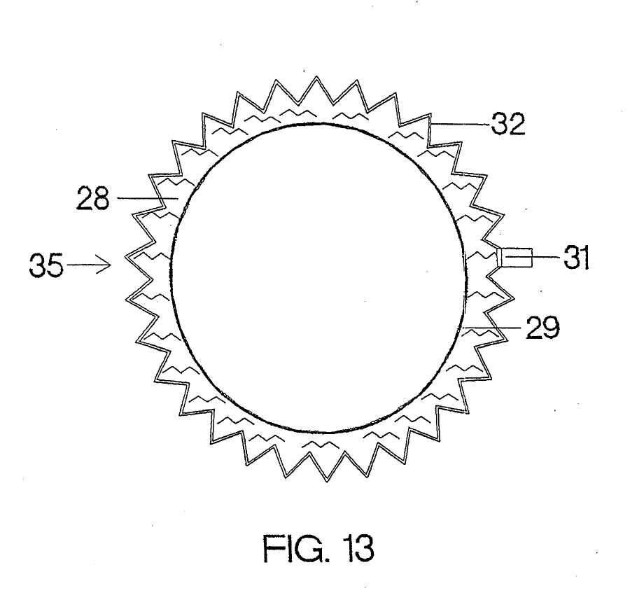

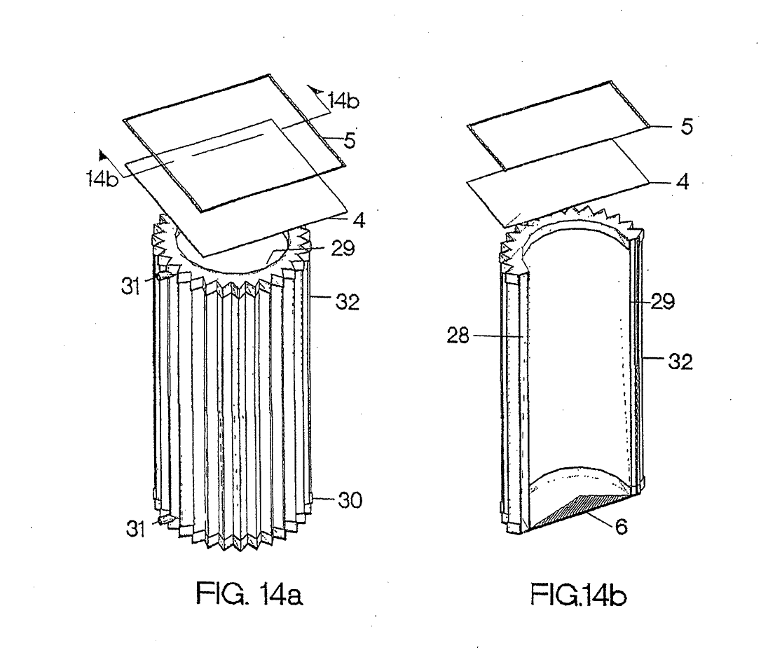

In an alternative embodiment wherein the numeral 35 is utilized to designate or represent a series of alternative embodiments of the growth units 1, the growth tube 3 is replaced by a growth cavity 28 as depicted in FIGS. 9-14. The growth cavity 28 is bounded on the interior side by a transparent or translucent, inner surface 29, a reflective surface 6 on the outer side, and two end caps 30 on the top and bottom, respectively as shown in FIGS. 10a and 10b. This alternative embodiment 35 is proportioned and shaped in a manner similar to the embodiment described above. The end caps 30 have ports 31, which function in the same manner as the two ends of the growth tubes 3; and radiation passes through a radiation diffusing component 4 and a radiation filter 5 in the same manner as described above with respect to the growth unit 1.

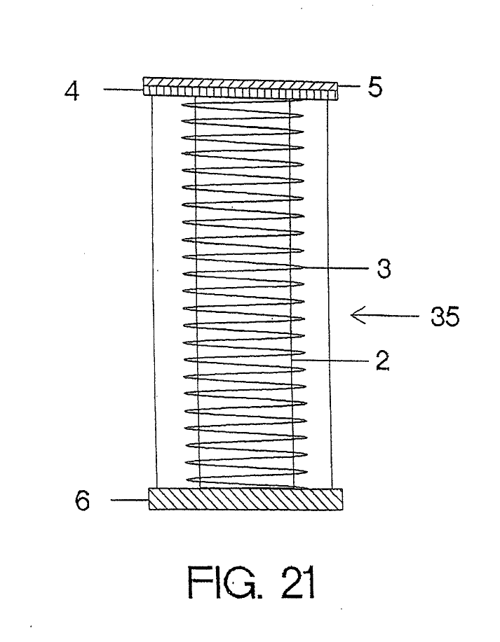

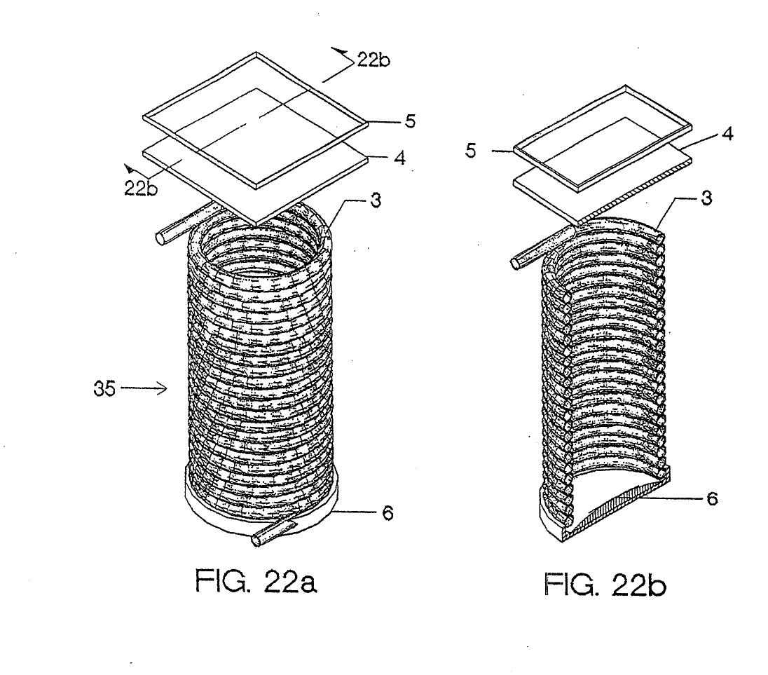

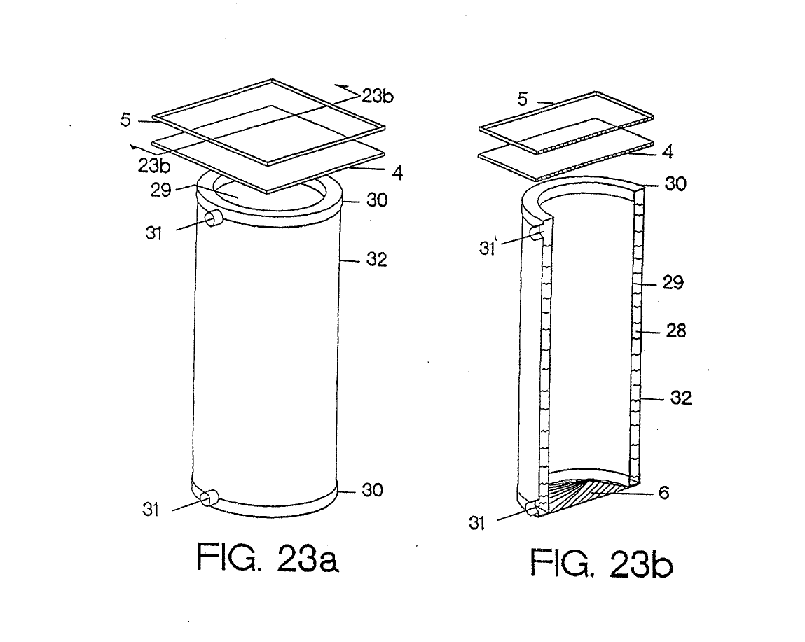

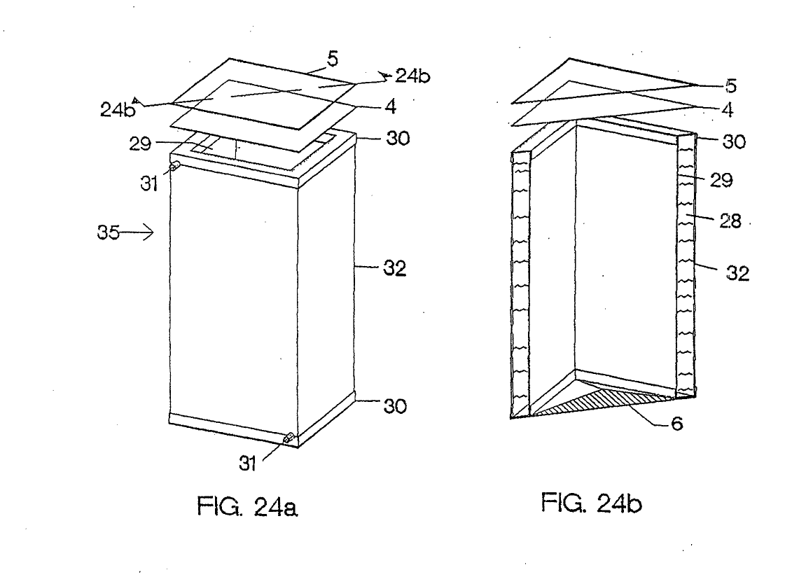

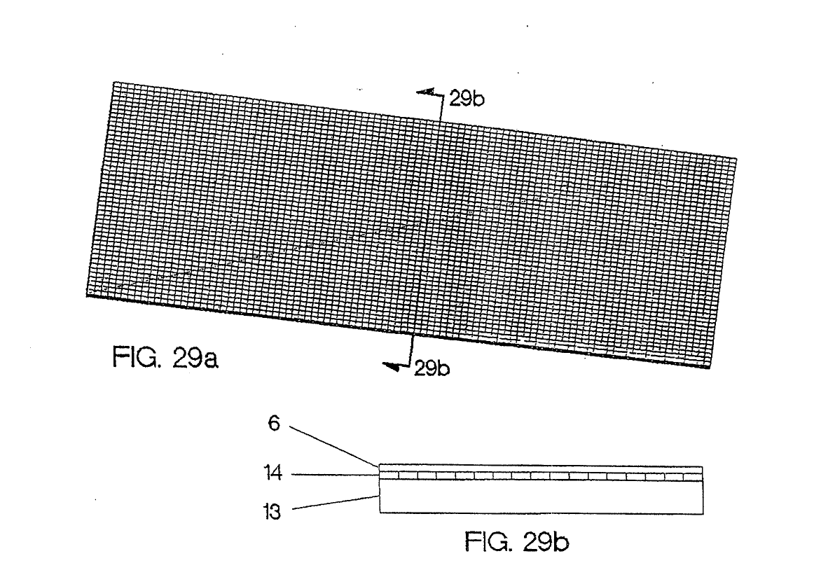

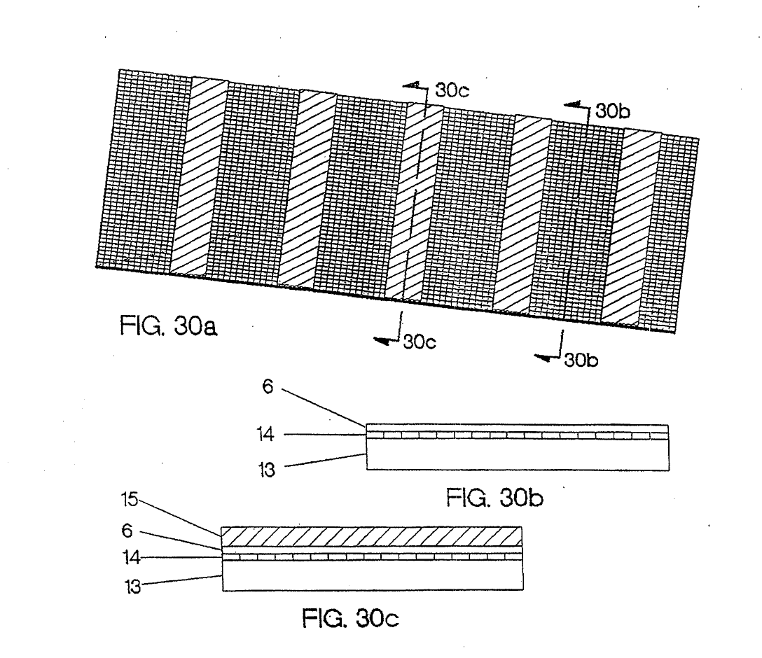

Other alternative embodiments of the invention are shown in FIG. 21-24. In this set of embodiments, the outer surfaces 32 of the growth units 1 are made of transparent or translucent material, rather than reflective material; and the interior wall panels shown in FIGS. 29a, 29b, 30a, 30b of the modules 22/housing 11 are lined with a reflective material on surface 6. In addition, the bottom 6a of module 22/housing 11 may also be reflective.

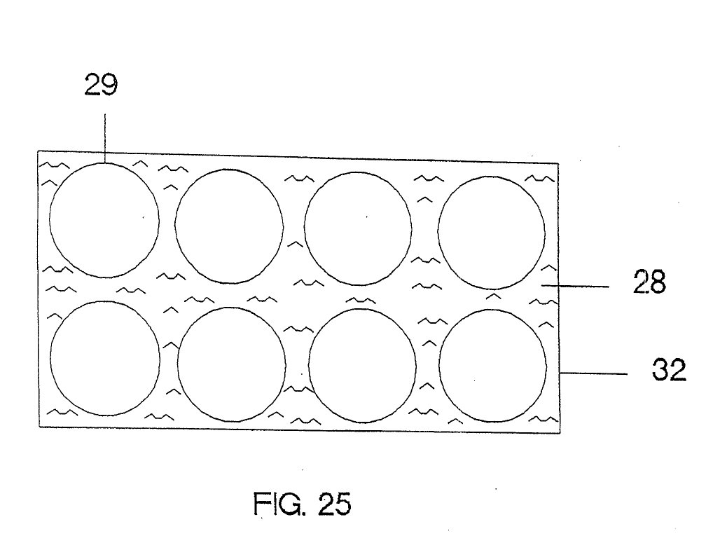

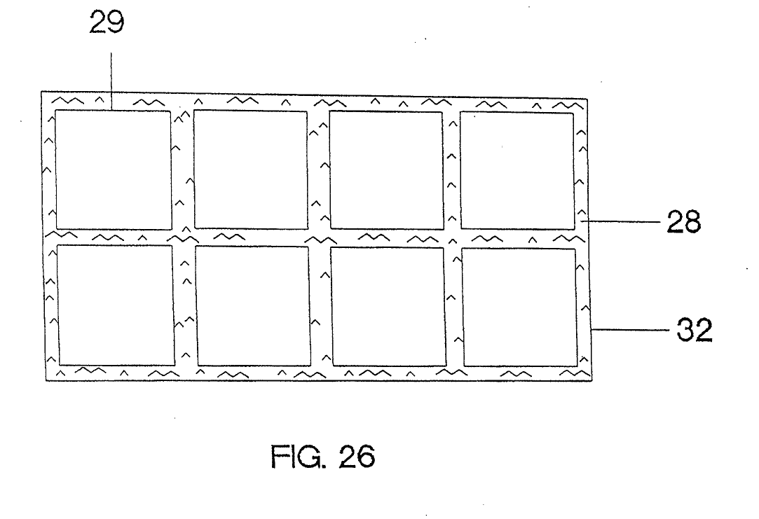

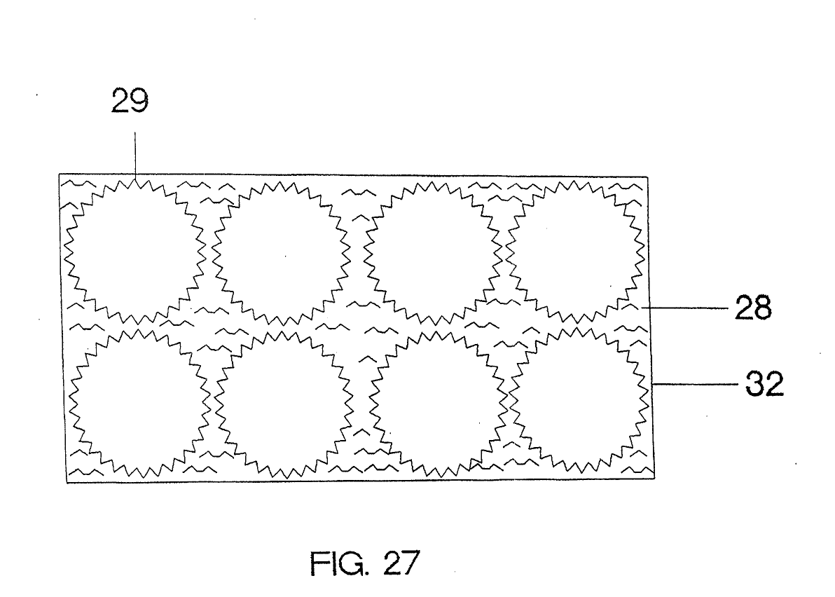

In yet another set of embodiments depicted in FIGS. 25, 26, & 27, the growth cavity 28 is a single, continuous cavity that extends all the way to the walls of the modules, rather than being separate cavities for each growth unit 1. In this set of embodiments, the inner surfaces 29 create radiation channels through which radiation is spread throughout a continuous growth cavity 28 rather than in separate growth cavities, as in other embodiments.

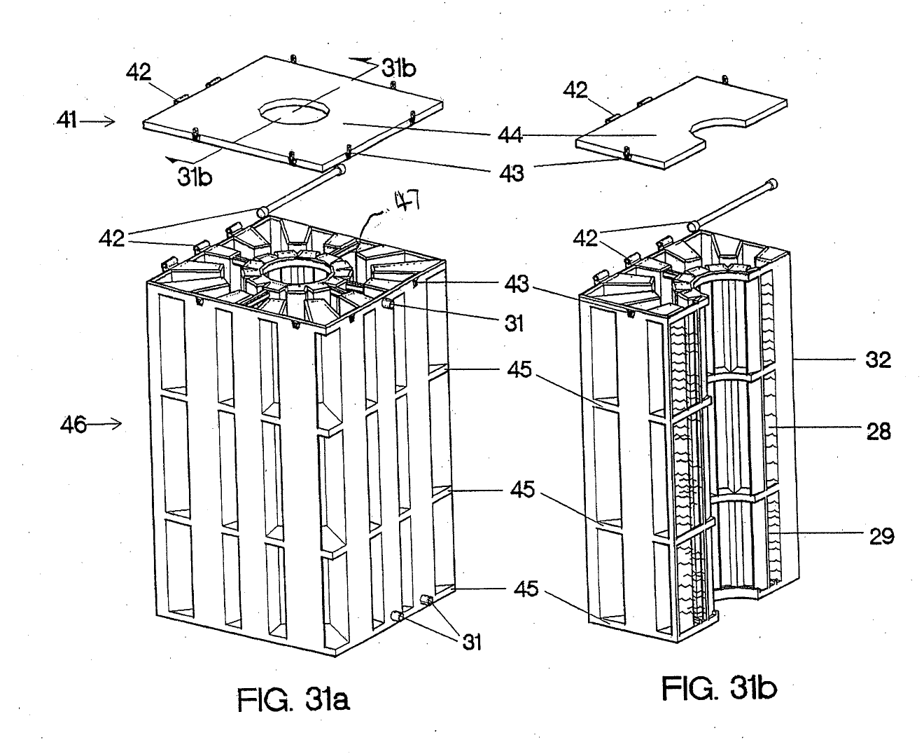

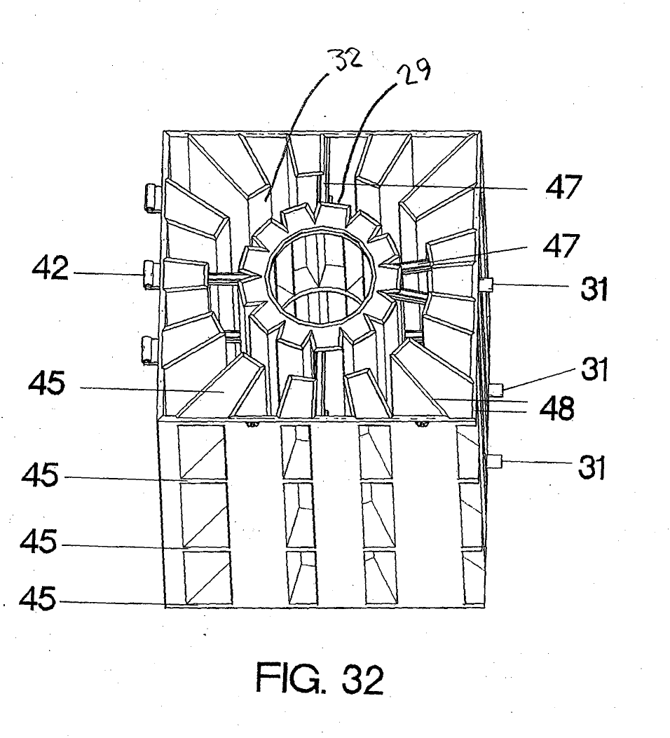

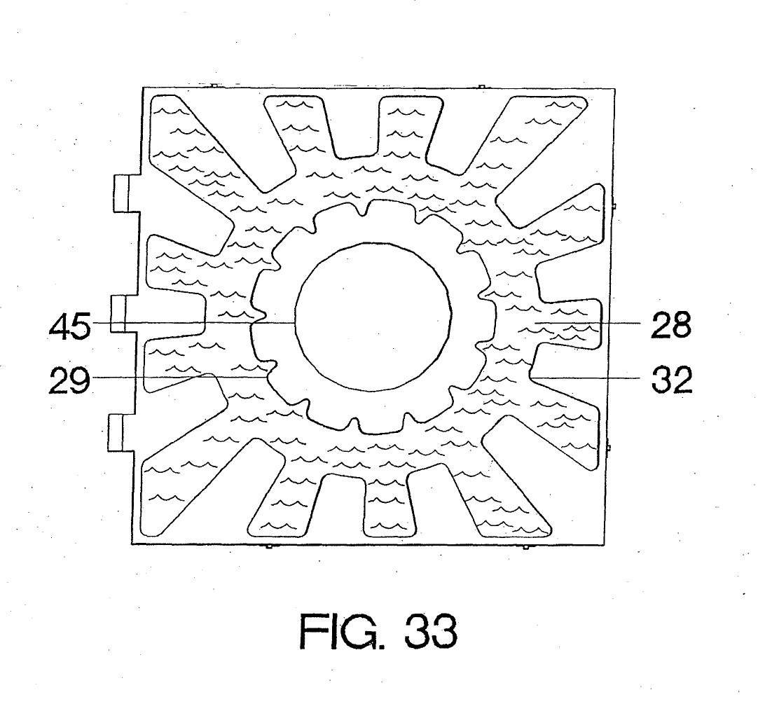

In still another embodiment of this invention, FIGS. 31a and 31b, 32, & 33 represent different views of the alternative embodiment of a single MOPS growth unit 46 in which substantially similar components of previous embodiments may be represented by similar reference numerals. More specifically, as shown in FIGS. 31a and 31b, the interior portion of unit 46 includes outer surfaces 32 and inner surfaces 29, both of which may be made of a material which permits radiation to pass therethrough and are formed of jagged and folded edges and surfaces in which the outer surfaces 32 may include indented portions of the wall of the unit 46 to form the jagged and protruding portions. In addition the central portions interior surfaces 29 may be formed of a series jagged or zig-zag sections as shown more clearly in FIGS. 32 and 33. These jagged, folded or zig-zagged surfaces, although other similar configurations may be used, provide an increase in respective surface areas, thereby exposing a greater number of growing algae cells to the radiation as well as an end cap (not shown). In addition, the surface adjacent at least the inner surface or member 29, which may be the bottom surface of the housing 11, may be reflective.

These drawings also show several additional features of a growth unit 46, including connecting components such as hinges 42 and fastening components such as clasps 43 to facilitate opening and cleaning of a growth unit 46. In addition, structural ribs 45 and structural braces 47, may be used to provide structural and dimensional strength and stability to the unit 46. Three ports 31 are also shown, although not limited to this number, are used for gas input, liquid input, and liquid harvest.

FIG. 33 is a top-down view of this embodiment, which shows the shape of the growth cavity 28, including the increased surface areas of the inner surfaces 29 and the outer surfaces 32, as well as the structural ribbing 45 of the growth unit. Preferably, though not limiting, this growth unit 46 may be fabricated with two plastic injection molds, one for the main body of the growth unit 46, (including the inner surfaces 29, the outer surfaces 32, and the end cap (not shown), the structural ribbing 45, the structural braces 47, the ports 31, and the hinges 42; and one for the hinged top cap 41. Also shown in FIG. 32 is a sealing member or agent such as gasket seal 48—preferably made from a sealant rubber material, though not limiting thereto which creates a substantially airtight seal when the hinged top cap 41 is closed.

The increased surface areas of the inner surfaces 29 and the outer surface 32 substantially increases the growth cavitiy's exposure to radiation. For example, but not limited thereto, a surface area of the inner and outer surfaces 29 and 32 (SA) with respect to the footprint area of the surface or ground upon which the growth unit rests (FP) may be a ratio SA:FP having a range of approximately 5:1 to 100:1. These ratios being examples only and do not limit the scope of this invention.

### Mode of Operation

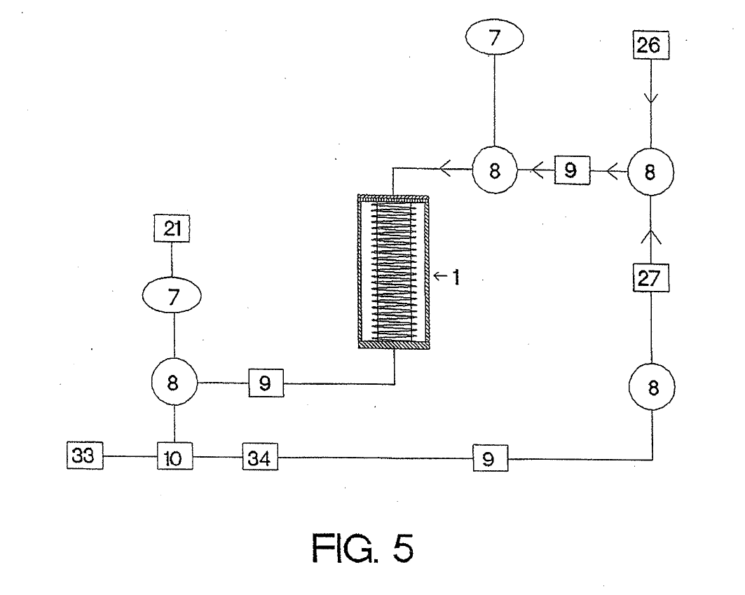

The following mode of operation is described with reference to FIGS. 5-8:

1. Seed Phase:

Now referring to FIG. 5, the MOPS system is “seeded” with appropriate contents. A small amount of live organisms, water, and the appropriate nutrient mixture are seeded into the growth tube 3 or growth cavity 28 by pumping them from the seed chamber 26 and the nutrient chamber 27 through 2-way valve 8 and pump 9. A wide variety of microorganisms can be grown in the MOPS, including photosynthetic bacteria and photosynthetic algae. In one embodiment, the algae species Cyanophyceae Genus Oscillatoria (a.k.a OSCIL2) is used, but there are many others that may also prove to be profitably grown in the MOPS. The specific formulation for nutrients will depend on the exact species of microorganism that is being grown. In one embodiment, although not limited thereto, a nutrient solution called, “SERI Type I” which comprises CaCl2, MgCl₂-6H₂O, Na₂SO₄, KCl, NaHCO₃, NaCl, & CaSO₄ may be used.

2. Growth Phase:

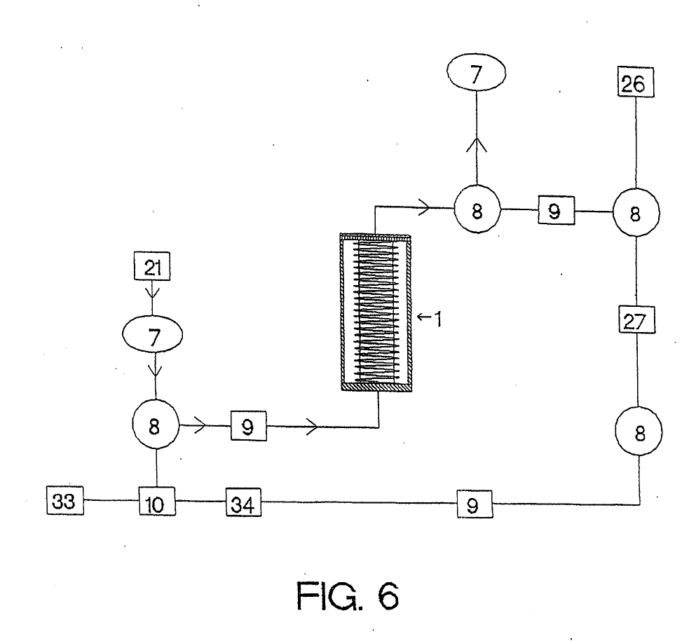

Now referring to FIG. 6, while the seeded tube(s) 3 or cavity(ies) 28 stands in the sunlight, filtered CO₂ from source 21 is diffused through the growth tube 3 or growth cavity 28 by way of filter 7, valve 8, and pump 9. The CO₂ source 21 can be any one of several sources, including: atmospheric air, bottled CO₂, or emissions from a power plant or other industrial source. It is desirable for the CO₂ source 21 (and its corresponding emissions from the top of the system) to be filtered with a very fine air filter 7, in order to prevent contamination of the system, by simply attaching two very fine air filters 7 onto either end of the system, as shown in the drawings, or by other equivalent means.

As the CO₂ is diffused through the growth unit 1, radiation strikes the surface of the growth tube 3 or growth cavity 28; and the micro-organism is simply allowed to grow for a period of time until the entire growth tube 3 or growth cavity 28 becomes opaque with algae or until such other time as harvest is desirable or profitable or otherwise chosen by the operator or by appropriate sensors.

Generally, it will be advantageous to utilize a fairly short growth cycle, in order to produce optimum yields, productivity, and profitability of the MOPS. If allowed to grow for too long, before harvesting and reloading the system, the algae solution will become thick with algae, which will reduce the productivity. Although it may seem counter-intuitive to harvest the solution before it becomes very thick with algae, approximately 3 days, although not limited thereto, may be used for maintaining a high rate of algae growth on a consistent basis.

3. Harvest Phase:

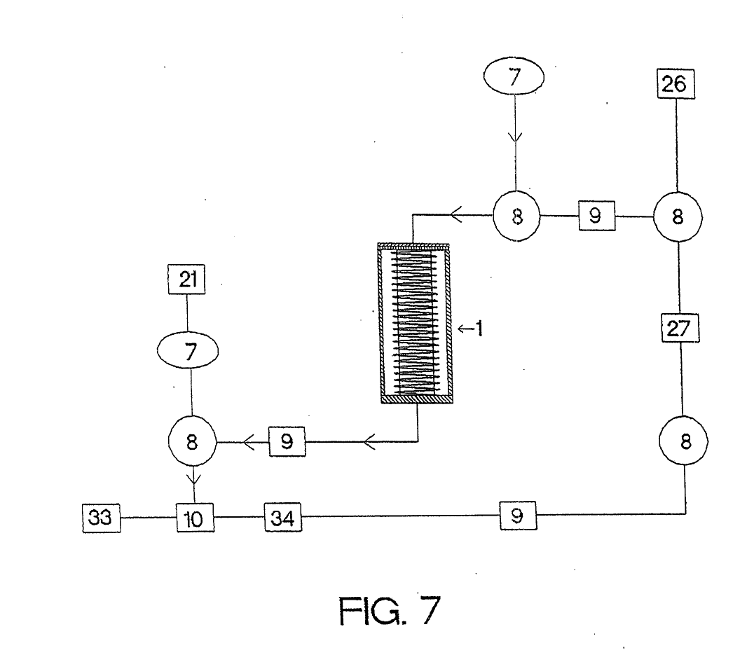

Now referring to FIG. 7, the CO₂ source 21 is shut off during the harvest phase, and the entire contents of the growth tube 3 or growth cavity 28 are pumped out of the growth tube via pump 9 into the chamber of the centrifuge 10 via valve 8, or into some other intermediary collection device prior to being transferred into the centrifuge 10.

4. Drying Phase:

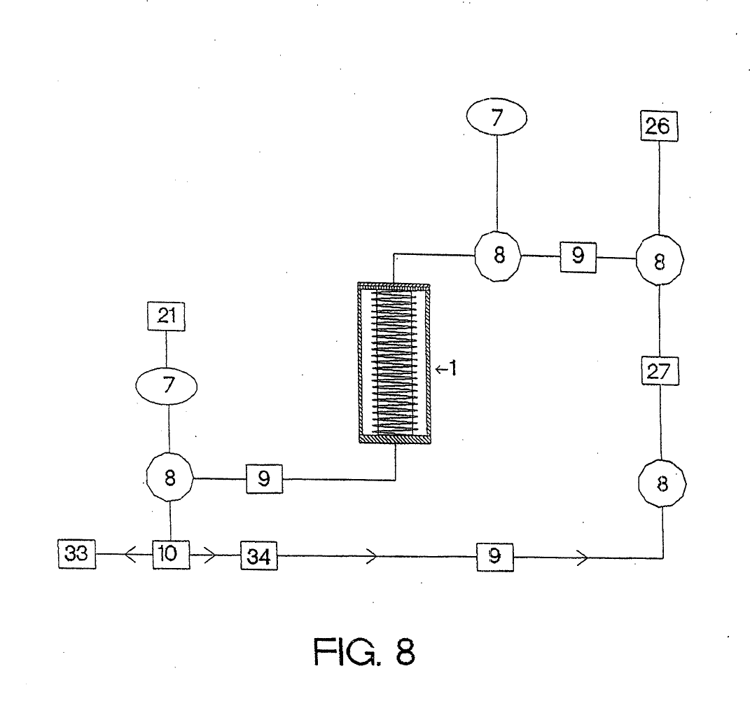

Now referring to FIG. 8, the liquid solution of micro-organisms is spun-dried in the centrifuge 10, in order to remove the water from the solution, leaving behind “dried micro-organisms” which may also be referred to as “dried algae” or by other names, which is the final product of the MOPS. Residual water is produced during the drying phase and is collected in a collecting component 34 and then recycled back into the nutrient chamber 27 via a pump 9 and a valve 8.

Although the invention has been described with respect to various embodiments, it should be realized that this invention is also capable of a wide variety of further and other embodiments within the spirit and scope of the appended claims.

What is claimed is:

1. A micro-organism production apparatus, comprising:
an inner member having a first end and a second end;
an outer structure circumscribing said inner member, said outer structure having a predetermined configuration facing said inner member;
a growth cavity formed between said inner member and outer structure capable of maintaining within said growth cavity micro-organisms in solution;
a radiation transmitting component adjacent at least said first end of said inner member capable of directing incoming radiation onto said growth cavity;
said predetermined configuration of said outer structure comprising means for substantially increasing its surface area, said surface area located on the interior portion of the outer structure facing said growth cavity;
wherein upon the receipt of a predetermined amount of live organisms, liquid, and nutrients within said growth cavity and the application of radiation through said radiation transmitting component, the apparatus produces the rapid growth of micro-organisms within said growth cavity.

2. The micro-organism production apparatus as defined in claim 1 wherein said means for substantially increasing the surface area of said outer structure comprises a series of protrusions and indentations.

3. The micro-organism production apparatus as defined in claim 2 wherein series of protrusions and indentations are in the form of jagged or zig-zag sections

4. The micro-organism production apparatus as defined in claim 2 further comprising at least one brace between said inner member and said outer structure.

5. The micro-organism production apparatus as defined in claim 2 wherein the interior portion of the outer structure facing said growth cavity is reflective and said inner member is made of a material which permits radiation to pass therethrough.

6. The micro-organism production apparatus as defined in claim 2 wherein said outer structure is made of a material which permits radiation to pass therethrough and said inner member is made of a material which permits radiation to pass therethrough.

7. The micro-organism production apparatus as defined in claim 6 further comprising a reflective surface adjacent at least said second end of said inner member.

8. The micro-organism production apparatus as defined in claim 6 wherein a surface area of the inner member facing said growth cavity comprises means for increasing its surface area thereof.

9. The micro-organism production apparatus as defined in claim 8 wherein said means for substantially increasing the surface area of said inner member comprises a series of protrusions and indentations.

10. The micro-organism production apparatus as defined in claim 8 further comprising a reflective surface adjacent at least said second end of said inner member.

11. The micro-organism production apparatus as defined in claim 10 wherein said micro-organisms are algae.

12. The micro-organism production apparatus as defined in claim 1 further comprising:
a radiation filtering component being adjacent to or incorporated as part of said radiation transmitting component capable of permitting predetermined wavelengths of radiation to pass onto said growth cavity.

13. The micro-organism production apparatus as defined in claim 12 wherein said radiation transmitting component and said radiation filtering component are movable with respect to said support member.

14. The micro-organism production apparatus as defined in claim 6 wherein said inner member is made of transparent material.

15. The micro-organism production apparatus as defined in claim 1 wherein said radiation transmitting component is a diffusion component.

16. The micro-organism production apparatus as defined in claim 1 wherein said radiation transmitting component is a Fresnel lens.

17. The micro-organism production apparatus as defined in claim 1 wherein said radiation transmitting component is a diffractive or refractive element.

18. The micro-organism production apparatus as defined in claim 1 wherein said radiation transmitting component is a holographic element.

19. The micro-organism production apparatus as defined in claim 10 further comprising means for tracking a source of radiation.

20. The micro-organism production apparatus as defined in claim 10 wherein the surface area of said outer structure and said surface area of said inner member are of a predetermined sized surface area.

21. A micro-organism production system, comprising:
at least one micro-organism growth unit, said at least one growth unit comprising
an inner member having a first end and a second end;
an outer structure circumscribing said inner member, said outer structure having a predetermined configuration facing said inner member;
a growth cavity formed between said inner member and said outer structure;
said predetermined configuration of said outer structure comprising means for substantially increasing its surface area, said surface area located on the interior portion of the outer structure facing said growth cavity;
a radiation transmitting component adjacent at least said first end of said inner member capable of directing incoming radiation into said growth cavity;
means for providing carbon dioxide to said at least one micro-organism growth unit;
a two way valve and a pump interposed between said means for providing carbon dioxide and said at least one micro-organism growth unit;
means for providing live organisms and liquid and nutrients to said at least one micro-organism growth unit; and
another two way valve and another pump interposed between said means for providing live organisms and liquid and nutrients and said at least one micro-organism growth unit;
wherein upon the receipt of a predetermined amount of the live organisms, liquid, and nutrients within said growth cavity and the application of radiation through said radiation transmitting component, the system produces the rapid growth of micro-organisms within said growth cavity of said at least one growth unit.

22. The micro-organism production system as defined in claim 21 wherein said micro-organisms are algae.

23. The micro-organism production system as defined in claim 21 further comprising:
a radiation filtering component being adjacent to or incorporated as part of said radiation transmitting component and capable of permitting predetermined wavelengths of radiation to pass onto said growth cavity.

24. The micro-organism production system as defined in claim 21 wherein said inner member is made of transparent material.

25. The micro-organism production system as defined in claim 23 wherein said radiation transmitting component is a Fresnel lens.

26. The micro-organism production system as defined in claim 23 wherein said radiation transmitting component is a diffractive or refractive element.

27. The micro-organism production system as defined in claim 23 wherein said radiation transmitting component is a holographic element.

28. The micro-organism production system as defined in claim 23 wherein said radiation transmitting component is a diffusion component.

29. The micro-organism production system as defined in claim 20 further comprising a plurality of micro-organism growth units.

30. The micro-organism production system as defined in claim 20 further comprising a reflective surface adjacent at least said second end of said inner member.

31. The micro-organism production system as defined in claim 29 further comprising:
a housing substantially encompassing said plurality of micro-organism growth units.

32. The micro-organism production system as defined in claim 31 wherein said outer structure and said inner member of said at least one of said plurality of growth units permit transmission of radiation therethrough.

33. The micro-organism production system as defined in claim 32 wherein said housing includes at least sides and a bottom, and where at least one of said sides and said bottom are reflective.

34. The micro-organism production system as defined in claim 33 further comprising a system for directing radiation to said plurality of growth units.

35. The micro-organism production system as defined in claim 33 further comprising means for controlling feeding of live micro-organisms in said plurality of growth units.

36. The micro-organism production system as defined in claim 33 further comprising means for providing an auxiliary source of radiation from heat generated within said housing.

37. The micro-organism production system as defined in claim 33 wherein said radiation transmitting component and said radiation filtering component are movable with respect to said inner member.

38. A micro-organism production apparatus, comprising:
an inner member having a first end and a second end, said inner member being made of a material that permits radiation to pass there through;
an outer structure circumscribing said inner member, said outer structure having a predetermined configuration facing said inner member, said predetermined configuration being in the form of protrusions and indentations;
a growth cavity formed between said inner member and said outer structure capable of maintaining within said growth cavity micro-organisms in solution;
a radiation transmitting component adjacent at least said first end of said inner member capable of directing incoming radiation onto said growth cavity; and
a reflective component adjacent at least said second end of said inner member;
wherein upon the receipt of a predetermined amount of live organisms, liquid, and nutrients within said growth cavity and the application of radiation through said radiation transmitting component, the apparatus produces the rapid growth of micro-organisms within said growth cavity.

39. The micro-organism production apparatus as defined in claim 38 wherein said outer structure is made of a material that permits radiation to pass there through.
